(19) United States
(12) Patent Application Publication
(10) Pub. No.: US 2021/0322612 A1
(43) Pub. Date: Oct. 21, 2021

(54) SYSTEM AND METHOD FOR AUTOMATICALLY SANITIZING PUBLICLY ACCESSIBLE USER INTERFACES

(71) Applicant: John Orvil Dean, Cleveland, GA (US)

(72) Inventor: John Orvil Dean, Cleveland, GA (US)

(21) Appl. No.: 17/001,867

(22) Filed: Aug. 25, 2020

Related U.S. Application Data
(60) Provisional application No. 63/012,465, filed on Apr. 20, 2020.

(51) Int. Cl. A61L 2/24; G05B 19/05; G07C 9/00; A61L 2/10; A61L 2/22; H02S 40/38; H02J 7/35

(52) U.S. Cl. CPC A61L 2/24; G05B 19/05; G07C 9/00174; A61L 2/10; A61L 2/22; A61L 2202/15; H02J 7/35; G05B 2219/1133; A61L 2202/11; A61L 2202/122; A61L 2202/14; H02S 40/38

(57) ABSTRACT

Exemplary embodiments of a system and method for automatically disinfecting publicly accessible user interfaces are disclosed. Certain embodiments are configured such that a user of a publicly accessible user interface, such as a fuel pump nozzle or an ATM keypad, actuates a shield or application housing in order to access the user interface. When the user completes use of the interface, the application housing is returned to its original state that covers the user interface. At such time, UVC light emitting diode arrays within the housing are energized in order to apply ultraviolet germicidal irradiation (“UVGI”). Consequently, any pathogen left on the user interface by the user will be remediated, making the user interface safe for use by a subsequent user.

### BACKGROUND

The present invention relates to systems and methods for disinfecting or sanitizing equipment and, more particularly, to a novel system and method for sanitizing a publicly accessible user interface.

Viruses and other pathogens often find their way from one host to another when a potential host touches a contaminated surface. Even a virus that isn't easily transmitted through airborne means can spread rampantly throughout a population when people unknowingly touch contaminated surfaces. All it takes, for example, is one infected person to use a gas pump or an ATM machine in order to subsequently infect anybody and everybody who uses the pump or ATM thereafter.

Clearly, it is desirable and in the general public's interest to disinfect publicly accessible user interfaces like fuel pump nozzles, ATM machine keypads, and touchscreens. Even so, publicly accessible user interfaces are rarely sanitized and, as such, are common culprits for the spread of disease. Enlightened users of publicly accessible user interfaces may take it upon themselves to sanitize a suspect interface before using it, but their actions in doing so do little to ensure that the user interface won't be re-contaminated by the very next user.

And so, there is a need in the art for a system and method that provides for automatic and routine sanitization of publicly accessible user interfaces.

### SUMMARY

Exemplary embodiments of a system and method for automatically disinfecting publicly accessible user interfaces are disclosed. Certain embodiments are configured such that a user of a publicly accessible user interface, such as a fuel pump nozzle or an ATM keypad, actuates a shield or application housing in order to access the user interface. When the user completes use of the interface, the application housing is returned to its original state that covers the user interface. At such time, UVC light emitting diode arrays within the housing are energized in order to apply ultraviolet germicidal irradiation (“UVGI”). Consequently, any pathogen left on the user interface by the user will be remediated, making the user interface safe for use by a subsequent user. It is further envisioned that certain embodiments of the solution may leverage an electrostatic spray nozzle within the housing, in lieu of or in addition to the UVC arrays, to apply an electrostatically charged and atomized liquid disinfectant to the user interface and, in that way, remediate any pathogen residing on the user interface.

An exemplary system for automatically disinfecting publicly accessible user interfaces according to the solution comprises first and foremost an application housing configured to define a space over a publicly accessible user interface such as a fuel pump nozzle or a keypad. The application housing is operable to translate between an open state and a closed state such that when the application housing is in the open state the publicly accessible user interface is accessible to a user and when the application housing is in the closed state the publicly accessible user interface is inaccessible to a user. The system further comprises an electrical power source, one or more arrays of UVC light emitting diodes residing within the application housing and in electrical communication with the electrical power source, and an actuation sensor configured to recognize whether the application housing is in the closed state or the open state. The actuation sensor may be, but is not limited to being, in the form of an infrared sensor or a mechanical switch operable to make or break an electrical circuit between the power source and the one or more arrays. Advantageously, when the actuation sensor indicates that the application housing is in the closed state the one or more arrays are energized from the electrical power source in order to subject the publicly accessible user interface to ultraviolet germicidal irradiation and, conversely, when the actuation sensor indicates that the application housing is in the open state the one or more arrays are de-energized.

The exemplary system for automatically disinfecting publicly accessible user interfaces may further include a controller, such as but not limited to a programmable logic controller, configured to receive an electrical signal from the actuation sensor and, in response to the signal, cause the one or more arrays to be either energized or de-energized. The exemplary system may further include a timer component configured to set a duration for which the one or more arrays are energized and a lock component configured to lock the application housing in the closed state while the one or more arrays are energized.

In some embodiments, there may be a solar charging panel electrically coupled to the power source. The one or more arrays of UVC light emitting diodes when energized emit electromagnetic radiation with a wavelength from 10 nm to 400 nm and light with a frequency from 30 petahertz to 750 terahertz. And, the exemplary according to the solution may further comprise a reflective surface plate positioned such that UVC light emitted from the one or more arrays of UVC light emitting diodes is reflected back toward the publicly accessible user interface (such as may be useful and applicable when the publicly accessible user interface is in the form of a fuel pump nozzle so that UVC light that passes by the fuel pump nozzle is reflected back to surfaces on the underside of the handle which are not directly exposed to the diodes).

In another exemplary embodiment, a system for automatically disinfecting publicly accessible user interfaces includes an application housing comprising one or more stationary, raised structures configured such that the publicly accessible user interface is always physically accessible to a user. The exemplary embodiment may include an electrical power source and a liquid disinfectant reservoir for holding a chemical disinfectant. One or more arrays of UVC light emitting diodes reside within the one or more raised structures of the application housing and are in electrical connection with the electrical power source. Similarly, one or more arrays of spray nozzles reside within the one or more raised structures of the application housing and may be operable to impart an electrostatic charge to an atomized flow created and discharged from the nozzles. The spray nozzles, whether electrostatic in design or not, are in fluid connection with the disinfectant reservoir and in electrical connection with the electrical power source (or, at least, a solenoid or some other electromechanical component operable to control flow of disinfectant from the reservoir to the nozzle is in electrical connection with the power source). And an actuation sensor configured to recognize physical proximity of a user is also comprised within the system.

Advantageously, when the actuation sensor indicates that a user is in physical proximity the one or more arrays of UVC light emitting diodes and the one or more arrays of spray nozzles are deactivated and when the actuation sensor indicates that a user is no longer in physical proximity the one or more arrays of UVC light emitting diodes are activated in order to subject the publicly accessible user interface to ultraviolet germicidal irradiation and/or the one or more arrays of the spray nozzles are activated in order to apply a fog of liquid disinfectant to the publicly accessible user interface.

The exemplary system may further comprise a controller configured to receive an electrical signal from the actuation sensor and, in response to the signal, cause the one or more arrays to be either energized or de-energized. The controller may be a programmable logic controller configured to execute instructions stored in the controller. The exemplary system may further comprise a timer component configured to set a duration for which the one or more arrays are energized. The system may also include a solar charging panel electrically coupled to the power source. The actuation sensor may be a motion sensor in the form of an infrared sensor. The one or more arrays of UVC light emitting diodes when energized may emit electromagnetic radiation with a wavelength from 10 nm to 400 nm and light with a frequency from 30 petahertz to 750 terahertz. And, the publicly accessible user interface associated with the exemplary embodiment may be in the form of, but is not limited to being in the form of, a fuel pump nozzle, a charging cable head for an electric vehicle charging station, a keypad, a touchscreen, or an actuation button.

### BRIEF DESCRIPTION OF THE DRAWINGS

In the drawings, like reference numerals refer to like parts throughout the various views unless otherwise indicated. For reference numerals with letter character designations such as “111A” or “111B”, the letter character designations may differentiate two like parts or elements present in the same figure or related figures. Letter character designations for reference numerals may be omitted when it is intended that a reference numeral encompass all parts having the same reference numeral in all figures.

FIG. 1 is a functional block diagram illustrating various components that may be comprised within given embodiments of the solution for sanitizing publicly accessible user interfaces;

FIGS. 2A-2B illustrate an exemplary embodiment of the solution configured for sanitizing a publicly accessible user interface in the form of a fuel pump nozzle of a fuel pump dispensing station;

FIGS. 3A-3C demonstrate a method of use for the embodiment of the solution illustrated in FIG. 2;

FIG. 4A-4C demonstrate a method of use for an exemplary embodiment of the solution configured to sanitize a publicly accessible user interface in the form of a keypad;

FIG. 5 illustrates another exemplary embodiment of the solution configured for sanitizing a publicly accessible user interface in the form of a fuel pump nozzle, as well as other exemplary publicly accessible user interfaces, of a fuel pump dispensing station; and

FIGS. 6A-6C demonstrate a method of use for the fuel pump nozzle in the FIG. 5 illustration.

### DETAILED DESCRIPTION

Various embodiments, aspects and features of the present invention encompass a system and method for automatically sanitizing a publicly accessible user interface after each and every use, although it is not a requirement of all embodiments of the solution that sanitization of the publicly accessible user interface happen after each use. Examples of publicly accessible user interfaces include, but are not limited to, fuel pump nozzles, keypads, touchscreens, money dispensers, actuation buttons, biometric readers, etc. Although the exemplary embodiments of the solution shown and described herein are envisioned for fuel pump nozzles and ATM user interfaces and the like, the scope of the solution is not so limited. For example, it is envisioned that other embodiments of the solution, or even embodiments of the solution shown and described herein, may be configured for automatic disinfection of devices not usually considered to be a publicly accessible user interface.

In this description, the terms sanitizing and disinfecting, and their conjugations, are used interchangeably to refer to the functional goal of embodiments of the solution, namely, to mitigate the concentration of, if not altogether kill or remove, harmful pathogens from a target surface such as a publicly accessible user interface.

In this description, the term “pathogen” refers to any bacterium, virus, or other microorganism that can cause disease to humans and/or animals.

In this description, the terms “UV” and “UVC” are used interchangeably to refer to electromagnetic radiation with a relatively short wavelength from 10 nm to 400 nm. The frequency of UVC light is commonly understood to range from about 30 petahertz to 750 terahertz. As will become better understood from the following disclosure, certain embodiments of the solution may leverage arrays of UVC light-emitting diodes to automatically disinfect a target surface, such as a publicly accessible user interface, through application of ultraviolet germicidal irradiation (“UVGI”). Advantageously, application of the UVC light to a target surface works to destroy nucleic acids in pathogens present thereon and disrupts their DNA, leaving them unable to perform vital cellular functions.

In this description, the term “LED” and “LEDs” refer to UVC light-emitting diodes. Certain embodiments of the solution leverage an array, or arrays, of LEDs in order to sanitize and disinfect a publicly accessible user interface with UVGI.

In this description, the terms “spray,” “mist,” “fog” and the like are used interchangeably to refer to an atomized flow of chemical disinfectant such as, but not limited to, alcohol. Similarly, in this description the terms “electrostatic spray,” “electrostatic mist,” “electrostatic fog” and the like are used interchangeably to refer to an atomized flow of chemical disinfectant to which an electrical charge has been imparted at the time of atomization. As one of ordinary skill in the art of electrostatic spraying would understand, an electrically charged atomized flow may be advantageously attracted magnetically to the surface of both animate and inanimate objects within its exposure. In this way, a chemical disinfectant may be applied thoroughly and universally to the entire exposed surface of a given object. Accordingly, it is envisioned that certain embodiments of the solution may leverage electrostatic technology for automatic application of a chemical disinfectant to publicly accessible user interfaces.

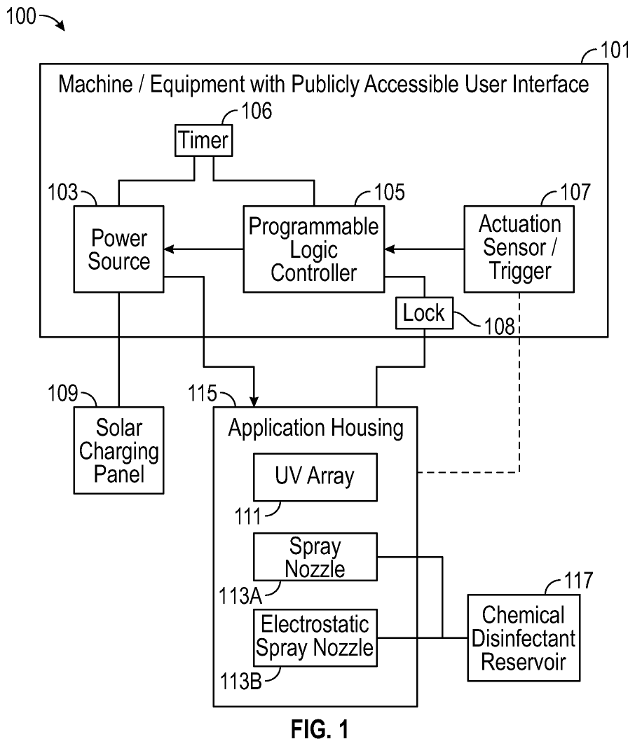

Referring to FIG. 1, shown is a functional block diagram illustrating various components that may be comprised within given embodiments of the solution 100 for sanitizing publicly accessible user interfaces. As non-limiting examples, a machine/equipment with publicly accessible user interface(s) 101 might be a fuel dispensing station (i.e., a “gas pump”) with a fuel pump nozzle and touchscreen and actuation buttons or, as another example, might be an ATM machine with a keypad and money dispenser. Other applications are envisioned and would occur to those with skill in the art and, as such, the scope of the solution is not limited to publicly accessible user interfaces specifically shown or referred to herein.

Returning to the FIG. 1 illustration, the machine with a publicly accessible user interface 101 may include power source 103. The power source 103 may be a “hard wired” power source or, alternatively, may be a rechargeable power source such as rechargeable batteries. If rechargeable in form, the power source 103 may be in electrical communication with a solar charging panel 109 or some other means of replenishing charge. Moreover, the power source 103 may include an AC to DC converter, a power management integrated circuit (“PMIC”), a switch operable to “make or break” the power supply, and/or other components and arrangements understood by those of skill in the art of electronics and electromechanics.

Notably, it is envisioned that some embodiments of the solution may be configured for retrofit to existing applications, such as to a fuel dispensing station, while other embodiments may be integrated into the given machine with user interfaces at the time of its manufacture. Moreover, depending on whether a given embodiment of the solution is configured for retrofitted applications or configured for integration into a given machine, the embodiment may leverage an existing power source 103 in the machine or may incorporate its own power source 103. As such, the location of the various components shown in the FIG. 1 illustration are for demonstration only and are not meant to imply or suggest that any given component in the overall system 100 must be within, or apart from, the machine 101 with a publicly accessible user interface.

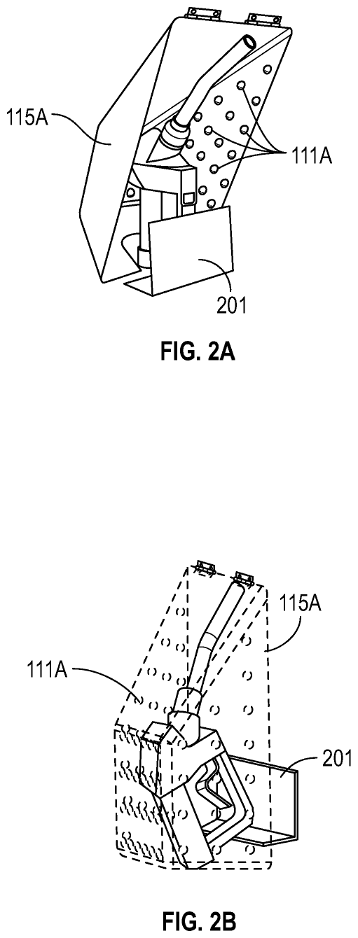

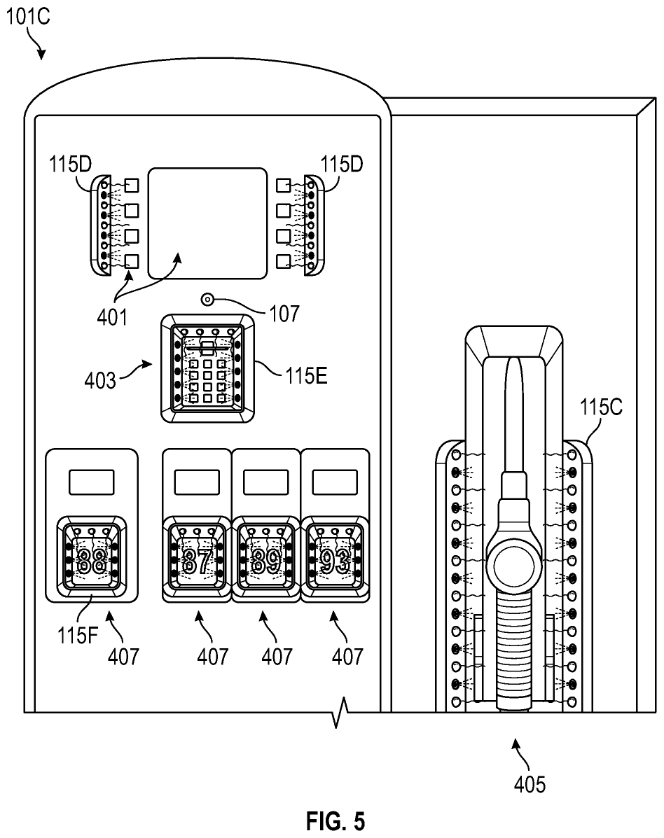

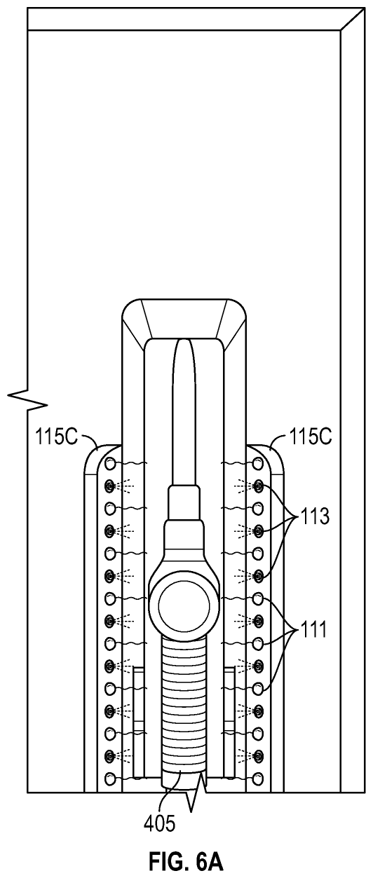

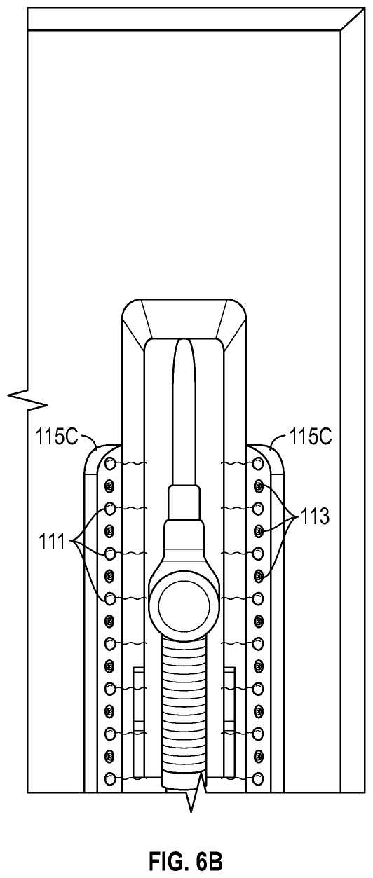

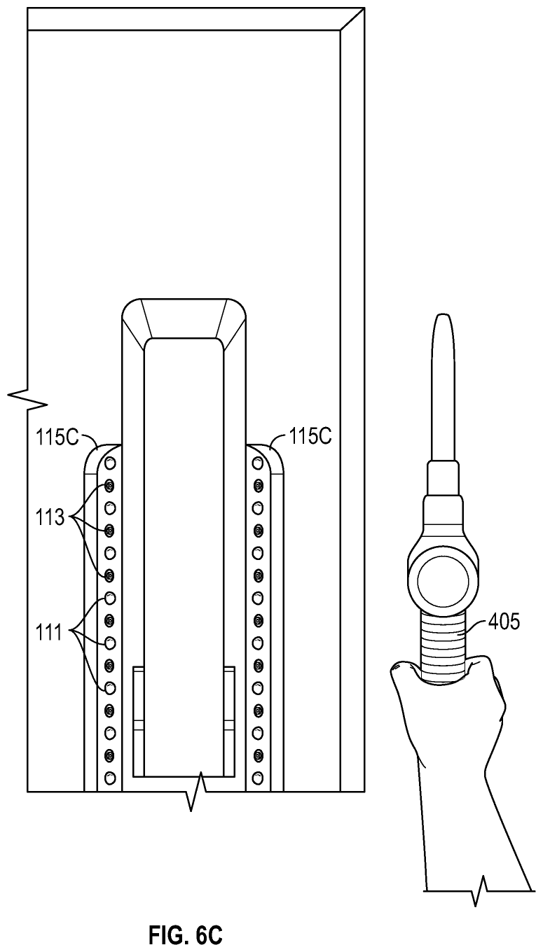

The system 100 may include an application housing 115 in the form of, for example, a shield or hood or clamshell configured to physically cover or envelope or enclose or partially enclose a publicly accessible user interface targeted for automatic disinfection. For example, the application housing 115 may be designed to cover a fuel pump nozzle or a keypad (see, for example, FIGS. 2-4). Or, similarly, the application housing 115 may be configured to partially cover or surround a given publicly accessible user interface, such as a “clamshell” design (see, for example, FIGS. 5-6).

The application housing 115 may incorporate one or more of a UV array 111, a spray nozzle(s) 113A, and an electrostatic spray nozzle(s) 113B. It is envisioned that a UV array 111 may comprise a plurality of LEDs configured to emit UVC light when the application housing 115 is applied over the publicly accessible user interface or when triggered to emit UVC light by some other means such as, but not limited, to an infrared motion sensor. Similarly, it is envisioned that a spray nozzle(s) 113A and/or an electrostatic nozzle(s) 113B may be configured to atomize (if required) and electrically charge (if an electrostatic nozzle 113B) a pressurized fluid flow of disinfectant from a reservoir 117. That is, a spray nozzle(s) 113A may emit a fog or heavy droplet spray while an electrostatic nozzle(s) 113B may emit an electrically charged, atomized spray. The reservoir 117 may be refillable, depending on embodiment, and may be pressurized by any suitable means including, but not limited to, a positive displacement pump, a compressed air source, or an incorporated compressed gas (I.e., an aerosol). Notably, for those embodiments of a reservoir 117 that leverage a compressed gas in order to deliver the disinfectant to a nozzle 113 in aerosol form, the nozzle 113 may not be configured for atomization of the fluid flow.

Returning to the FIG. 1 illustration, a user of the publicly accessible user interface may lift or mechanically translate the application housing 115 from a closed state to an open state in order to physically access the user interface. In doing so, an actuation sensor or trigger 107 may recognize the movement of the housing 115 and provide an electrical signal to a controller 105. It is envisioned that the actuation sensor or trigger 107 may be in the form of, but not limited to, an infrared sensor, a spring-loaded normally open mechanical switch, a magnetic switch, etc. The transition of the housing 115 from a closed state to an open state, and vice versa, may trigger the sensor 107 or actuate the switch 107, depending on the form of the sensor/trigger 107. The controller 105 may perform as a “soft switch” in response to the electrical signal from sensor 107 and “make” or “break” a power supply circuit from power source 103 to the UV array(s) 111 of application housing 115. The controller 105 may also leverage a timer 106 for controlling how long the array(s) 111 stay energized after the application housing 115 is returned to a closed state. Similarly, the controller 105 may leverage an automatic lock 108 in order to prevent the application housing 115 from being opened by a next user until the UV array(s) 111 have completed UVGI and are no longer energized. It is envisioned that the controller 105 may be in the form of a programmable logic controller (“PLC”), as would be readily recognized by those of skill in the art of programming, however, other types and forms of controllers suitable for specific applications will occur to those of skill in the art and, as such, the controller 105 is not limited to the form of a PLC.

Further to that which is envisioned above, the actuation sensor or trigger 107 in some embodiments may comprise a switch such that when the application housing 115 is “up” and in an open state the switch “breaks” a power supply circuit to the application housing 115, thereby causing the UV array(s) 111 to cease UVC light emission, and when the application housing 115 is “down” and in a closed state over the user interface the switch “makes” the power supply circuit to the application housing 115 thereby causes the UV array(s) 111 to generate UVC light. In such an embodiment, the PLC 105 may not be required. It is envisioned that in such embodiments that forego use of a “soft switch” through a PLC 105 in favor of a mechanical switch in actuation sensor/trigger 107 that directly makes or breaks a power circuit supplied by power source 103, the UV array(s) 111 may remain constantly powered and producing UVC light when the application housing 115 is “down” in its closed state and covering the user interface.

In still other embodiments, a timer 106 may work in conjunction with controller 105 and/or actuation sensor/trigger 107 in order to dictate an amount of time for which the power supply 103 may supply power to the UV array(s) 111 when the application housing 115 is in a “down” position covering the publicly accessible user interface. For example, embodiments with timers 106 may be configured such that the UV array(s) 111 are powered on periodically without regard for whether the application housing 115 is in an “up” state or “down” state. As another example, embodiments of the solution with timers 106 may be configured such that the UV array(s) 111 are powered and emitting UVC light for a set duration of time when the sensor 107 indicates that the application housing 115 is in a down position, after which the power supply circuit from the power source 103 to the UV array 111 is broken in order to cause the LEDs of the array 111 to cease UVC light generation.

As previously disclosed, it is envisioned that in some embodiments of the solution the application housing 115 may not be configured to transition between open and closed states. In such embodiments, the application housing 115 may take the configuration of a clamshell or some other physical structure that directs the LEDs 111 and/or the nozzles 113 toward the user interface without preventing user access to the user interface. Examples of such an arrangement for an application housing 115 can be see in the FIGS. 5-6 illustrations that follow. For such embodiments, the system 100 may include an infrared or motion sensor 107 that is operable to detect the physical presence of a user near the system 100. A signal generated by the sensor 107 may be interpreted by the controller 105 to indicate when the UV arrays 111 may be powered and/or when the nozzles 113 may be actuated. In this way, an embodiment of the solution that leverages an “always open” structure for an application housing 115 may be configured such that the LEDs are not powered and/or the nozzle(s) 113 are not actuated when a user is in near vicinity to the system 100. It is further envisioned that embodiments of the solution that include “always open” application housings 115 may leverage a timer 106 and executable logic stored in controller 105 to dictate when and for how long UV arrays 111 may be powered and/or nozzle(s) 113 may be actuated.

For those applications of the solution that leverage an application housing 115 configured to translate between and open state and a closed state (as opposed to those that are “always open”), when the user has completed use of the user interface the user may return the application housing 115 to its original state, i.e. to a “closed” state that covers the user the interface. In doing so, in certain embodiments the sensor 107 may recognize the change of physical position of the application housing 115 and provide a signal indicating such to the controller 105, as previously described. In turn, the controller 105 may cause the power source 103 to supply electrical energy to the application housing 115 for a certain period of time in order to actuate the UV array 111 and/or the spray nozzle(s) 113. In this way, any pathogen that may have been imparted to the publicly accessible user interface by the user's use will be remediated. Advantageously, the publicly accessible user interface is disinfected and ready for use by the next user.

The proper amount and duration of UVC light that may be applied to a given publicly accessible user interface will occur to those of skill in the art. Similarly, the volume of disinfectant spray, whether atomized and/or charged, that may be applied to a publicly accessible user interface by an embodiment of the solution that leverages an electrostatic spray nozzle 113B and/or a non-electrostatic spray nozzle 113A will also occur to those with skill in the art.

It is envisioned that the actuation sensor/trigger 107 may be in the form of a mechanical switch that “makes or breaks” an electrical circuit when the application housing 115 is physically translated from one position to another by a user wishing to access a publicly accessible user interface, as described above in more detail. It is further envisioned that the actuation sensor/trigger 107 may be in the form of an electrical sensor, such as an infrared sensor, that recognizes movement of the application housing 105. It is further envisioned that the actuation sensor/trigger 107 may be in the form of an electrical sensor, such as an infrared sensor, that recognizes movement indicating the physical presence of a user. Useful and proper sensor types and configurations for actuation sensor/trigger 107 will occur to those of skill in the art.

FIGS. 2A-2B illustrate an exemplary embodiment of the solution configured for sanitizing a publicly accessible user interface in the form of a fuel pump nozzle of a fuel pump dispensing station. FIG. 2A is an inside perspective view of the embodiment showing the application housing 115A in a closed state over the fuel pump handle. The application housing 115A is an example of an application housing configured to transition from a “closed state” where it fully encloses the user interface to an “open state” where it allows for user access to the user interface. Similarly, FIG. 2B is an outside perspective view of the same embodiment with the application housing 115A illustrated in a clear, “see-through” form.

In the FIG. 2 illustrations, the application housing 115A is configured like a hood to cover a fuel pump nozzle and comprises arrays of UVC-emitting LEDs 111A. The embodiment further includes a reflective surface plate 201 strategically placed such that UVC light emitted from the diodes 111A may be advantageously reflected back toward the fuel pump nozzle. Consistent with that which has been described above, a user may lift the application housing 115A in order to access the fuel pump nozzle. Upon return of the fuel pump nozzle to its cradle, the application housing 115A may be returned to its closed state (as depicted in the FIG. 2 illustrations) and, accordingly, the sensor 107 may alert the controller 105 to energize the arrays 111A. Alternatively, and depending on embodiment, the timer 106 may dictate how long the arrays 111A are energized after the application housing 115A is returned to the closed state. Notably, the timer function 106 may be in the form of a software algorithm comprised within the controller 105, depending on embodiments. The automatic lock mechanism 108, if present, may actuate in order to prevent a next user from lifting the application housing 115A until after the arrays 111A have been energized long enough to complete UVGI.

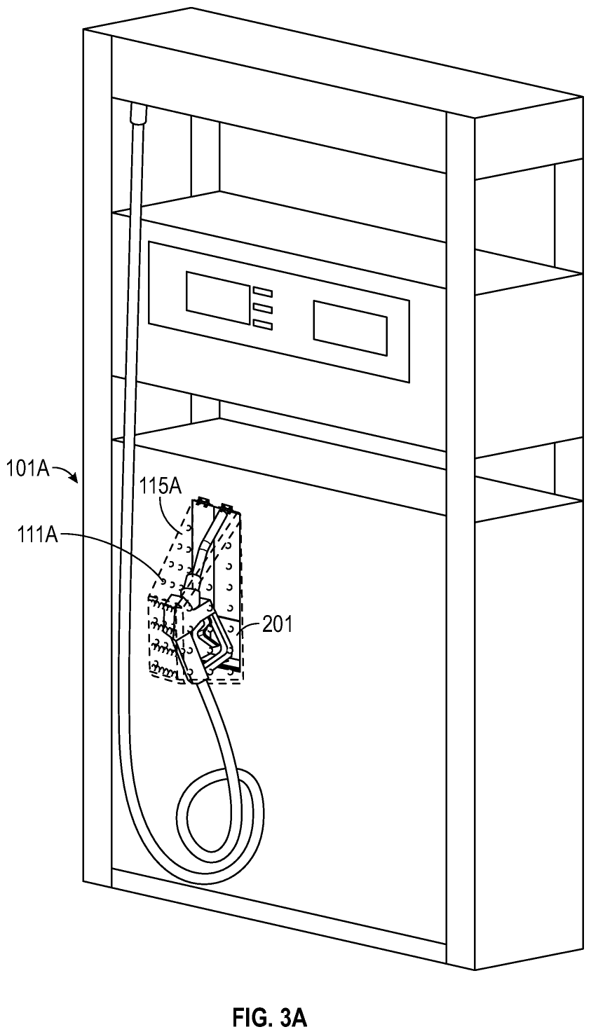

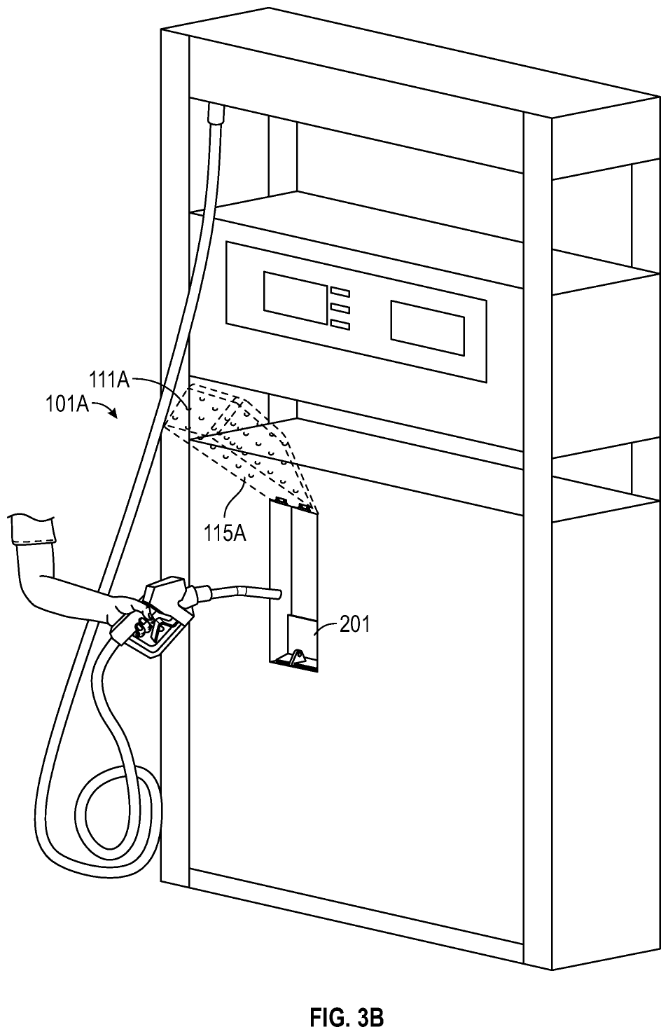

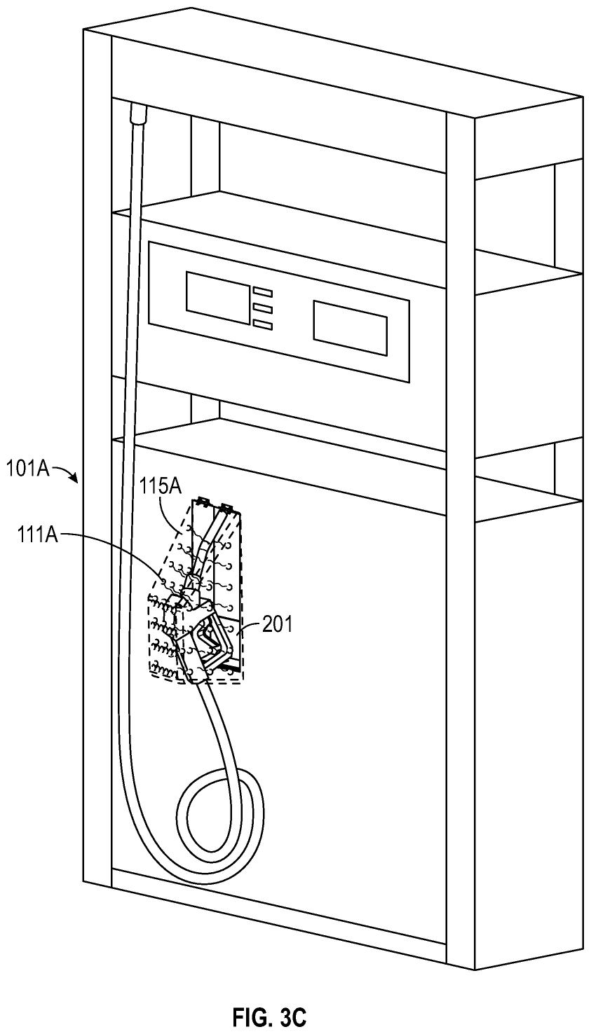

FIGS. 3A-3C demonstrate a method of use for the embodiment of the solution illustrated in FIG. 2. In the FIG. 3A illustration, the application housing 115A is in a closed state over the fuel pump nozzle of a fuel dispensing station 101A. When in this state, the UV array(s) 111A may have already been energized for a sufficient duration of time to accomplish the necessary UVGI and may have been de-energized according to a timer 106. A lock mechanism 108 may have been activated while the UV array(s) 111A were energized, in order to prevent interruption of the sanitization process by a next user lifting the housing 115A, and subsequently deactivated in order to allow a next user to access the fuel pump nozzle.

Moving to the FIG. 3B illustration, a next user has lifted the application housing 115A to an open state in order to access the fuel pump nozzle. Notably, because the UV array(s) 111A may have been previously energized for a sufficient duration to accomplish UVGI, the fuel pump nozzle may be sanitized and free of pathogens. The user has removed the fuel pump nozzle from its cradle and, in doing so, may have contaminated the fuel pump nozzle with new pathogens.

Turning to the FIG. 3C illustration, the user depicted in FIG. 3B has returned the fuel pump nozzle to its cradle and lowered the application housing 115A to its closed state over the fuel pump nozzle. Consequently, the actuation sensor/trigger 107 may have recognized that the application housing 115A has been lowered and, in turn, signaled to the controller 105 and/or the lock 108 and/or the timer 106 accordingly. The lock 108 may respond by locking the application housing 115A in the closed position while the controller 105 and/or timer 106 work with the power source 103 to energize the UV array(s) 111A for a duration of time sufficient to sanitize the fuel pump nozzle. At the end of such duration, the power supply from the power source 103 to the UV array(s) 111A may be broken in order to de-energize the arrays 111A and the lock 108 may deactivate in order to allow a next user to lift the application housing 115A and access the fuel pump nozzle.

Notably, although the illustrations of FIG. 3 have been described to demonstrate a method of use for an embodiment of the solution that leverages UV array(s) 111A to sanitize the exemplary publicly accessible user interface in the form of a fuel pump nozzle, it is envisioned that similar alternative embodiments of the solution may leverage instead a spray nozzle 113A and/or an electrostatic spray nozzle 113B (an electrostatic spraying nozzle, in and of itself, is understood in the art and, therefore, a detailed depiction and description of how an electrostatic spray nozzle operates is not included herein). The method of use of the solution may be essentially the same as that which has been described above with the exception that a spray of disinfectant or an electrostatic fog of disinfectant may be used in lieu of energized UV array(s). It is further envisioned that in some embodiments, such as the exemplary embodiments of the solution that are illustrated in FIGS. 5-6, UVC array(s) 111 and spray nozzles 113 may both be leveraged for sanitizing a publicly accessible user interface. Advantageously, for those embodiments of the solution that include both UVC array(s) 111 and spray nozzle(s) 113, control algorithms stored in and executed by controller 105 may leverage one or the other or both depending on the detected application scenario.

The exemplary embodiment depicted in FIGS. 2 and 3 depict UV arrays 111A, however, it is envisioned that a spray nozzle 113A and/or an electrostatic spray nozzle 113B (see FIG. 1) may be mounted inside the application housing 115 and fluidly connected to a pressurized chemical disinfectant reservoir 117 and electrically connected to the power source 103. Optionally, the nozzle 113 may be mounted inside the receptacle for receiving the fuel pump nozzle while still being necessarily fluidly connected to a pressurized chemical disinfectant reservoir 117 and electrically connected to the power source 103. The chemical disinfectant reservoir 117 may be mounted external to the pumping station or internal to the pumping station.

The sequence of activation for an embodiment of the solution that leverages a spray nozzle 113A and/or an electrostatic spray nozzle 113B may be the same as that which is described above relative to the FIGS. 3A-3C illustrations. That is, translation of the application hood from an open state to a closed state may trigger actuation of the spray nozzle(s) 113 such that it generates a simple fog or an electrostatically charged fog of chemical disinfectant within the space defined beneath the application housing. As would be understood by one of ordinary skill in the art, an electrostatically charged fog of chemical disinfectant may completely surround and coat the surfaces of the user interface (such as a fuel pump nozzle), thereby disinfecting and sanitizing the interface. It is further envisioned that the action of lowering the application housing from an open state to a closed state over the user interface may be leveraged in some embodiments as a pumping action to generate pressure in the chemical disinfectant reservoir 117, although such is not required in all embodiments of the solution that may use chemical disinfectant to sanitize a given user interface. As one of ordinary skill in the art would understand, however, the action of a user closing the application hood down over the user interface may conveniently supply a force for actuating a linear cylinder or the like that imparts pressurized air into reservoir 117.

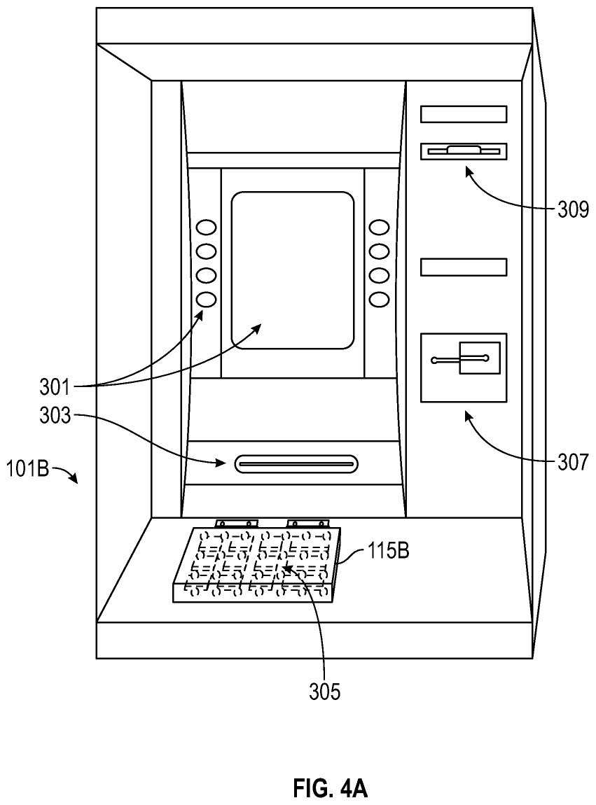

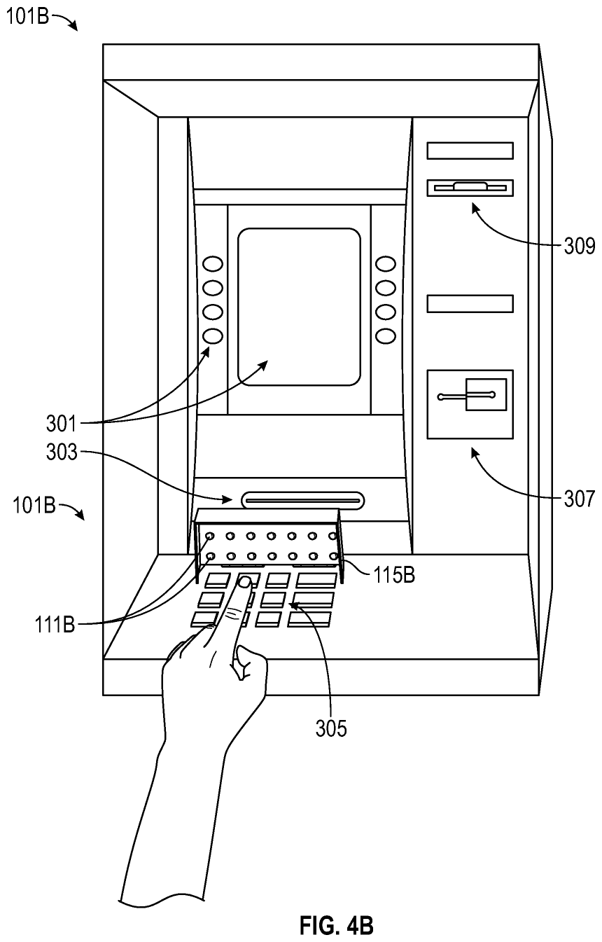

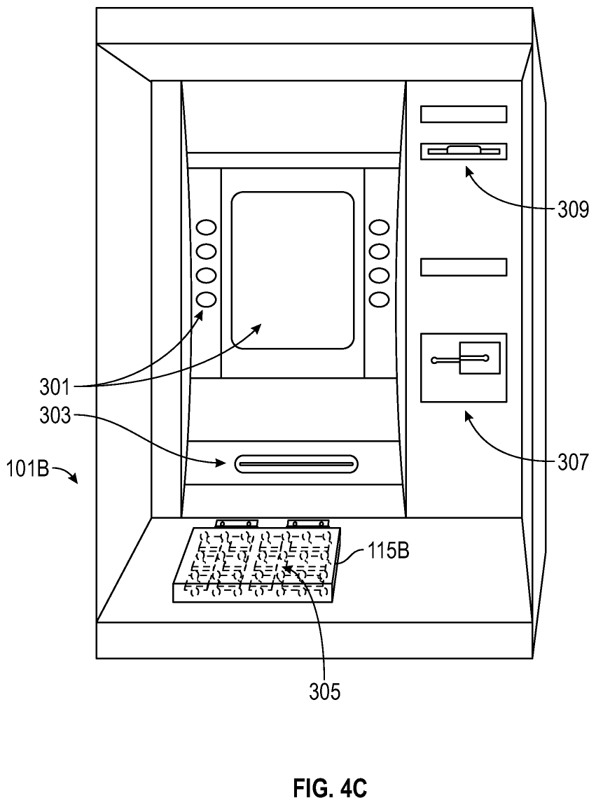

FIG. 4A-4C demonstrate a method of use for an exemplary embodiment of the solution configured to sanitize a publicly accessible user interface in the form of a keypad 305. The illustrations in FIGS. 4A-4C are based on an exemplary automated teller machine 101B that typically includes various publicly accessible user interfaces 301-309, any one or more of which may be sanitized by a particular embodiment of the solution configured therefor. As can be understood from the FIG. 4 illustrations, the publicly accessible user interface 301 may be in the form of a touchscreen including various touch-sensitive radio buttons. Consistent with that which has been described above, it is envisioned that an application housing 115B comprising UVC light arrays 111B may be configured to cover any one or more of the interfaces 301-309 such that its physical position translation triggers automatic application of UVGI.

The publicly accessible user interface 303 may be in the form of a money dispensing slot. Like general surfaces, money is known to be a common carrier of pathogens picked up from its being handled by many users over the course of commercial transactions. Consequently, it is envisioned that embodiments of the solution may incorporate UVC light arrays into the dispensing mechanism of an ATM 101B, thereby irradiating and sanitizing the money as it is dispensed to a user of an ATM 101B.

Similarly, the publicly accessible user interface 307 may be in the form of a token reader (e.g., a credit card or a debit card reader). Like general surfaces, credit tokens are known to be a common carrier of pathogens due to its being physically handled by its user. Consequently, it is envisioned that embodiments of the solution may incorporate UVC light arrays into the token reader of an ATM 101B, thereby irradiating and sanitizing the token as it is inserted into the reader by a user of an ATM 101B.

And, similarly, the publicly accessible user interface 309 may be in the form of a receipt dispenser. Although not representing quite as high a risk for transmission of pathogens as some other forms of publicly accessible user interfaces, it is envisioned that certain embodiments of the solution may incorporate UVC light arrays into the receipt dispensing mechanism of an ATM 101B, thereby irradiating and sanitizing the receipt as it is dispensed to a user of an ATM 101B.

Description of the FIG. 4 embodiment of the solution, however, will focus on the particular publicly accessible user interface 305 that is in the form of a keypad. Unlike the exemplary embodiment of the solution shown and described relative to the FIG. 2 and FIG. 3 illustrations, the particular user interfaces that may be found on an application like an ATM may not be well suited for repeat exposure to a fog disinfectant, whether electrostatically charged or not, and so it is envisioned that variations of embodiments that leverage UVC light arrays, such as has been described above, may be better suited for such applications.

Turning now to the method of use for the embodiment of the solution depicted in the FIG. 4A-4C illustrations, in the FIG. 4A illustration, the application housing 115B is in a closed state over the keypad 305. When in this state, the UV array(s) 111B may have already been energized for a sufficient duration of time to accomplish the necessary UVGI and may have been de-energized according to a timer 106. A lock mechanism 108 may have been activated while the UV array(s) 111B were energized, in order to prevent interruption of the sanitization process by a next user lifting the housing 115B, and subsequently deactivated in order to allow a next user to access the keypad.

Moving to the FIG. 4B illustration, a next user has lifted the application housing 115B to an open state in order to access the keypad 305. Notably, because the UV array(s) 111B may have been previously energized for a sufficient duration to accomplish UVGI, the keypad 305 may be sanitized and free of pathogens. The user has used the keypad 305 by pressing various keys and, in doing so, may have contaminated the keypad 305 with new pathogens.

Turning to the FIG. 4C illustration, the user depicted in FIG. 4B has lowered the application housing 115B to its closed state over the keypad 305. Consequently, the actuation sensor/trigger 107 may have recognized that the application housing 115B has been lowered and, in turn, signaled to the controller 105 and/or the lock 108 and/or the timer 106 accordingly. The lock 108 may respond by locking the application housing 115B in the closed position while the controller 105 and/or timer 106 work with the power source 103 to energize the UV array(s) 111B for a duration of time sufficient to sanitize the keypad 305. At the end of such duration, the power supply from the power source 103 to the UV array(s) 111B may be broken in order to de-energize the arrays 111B and the lock 108 may deactivate in order to allow a next user to lift the application housing 115B and access the keypad 305.

FIG. 5 illustrates another exemplary embodiment of the solution configured for sanitizing a publicly accessible user interface in the form of a fuel pump nozzle 405, as well as other exemplary publicly accessible user interfaces 401, 403, 407, of a fuel pump dispensing station 101C. As would be generally recognized by a user of a fuel dispensing station such as fuel dispensing station 101C, there are a number of publicly accessible user interfaces that may be physically contacted by a user—for example, a touchscreen with actuation buttons 401, a keypad 403, the fuel pump nozzle 405, and fuel grade/type selector buttons 407. Similarly, an electric vehicle charging station may include publicly accessible user interfaces similar to a typical fuel dispensing station as depicted in FIG. 5, albeit having a charging cable head instead of a fuel pump nozzle, and so it is envisioned that an EVC station may be an application for embodiments of the solution.

For the exemplary embodiment of a fuel dispensing station 101C demonstrated in the FIG. 5 illustration, the solution 100 includes an associated application housing 115 for each of the publicly accessible user interfaces 401, 403, 405, 407. The application housings 115C-F illustrated in the FIG. 5 embodiment are of an open configuration that surrounds the interface while allowing a user to access the interface. It can be seen that the application housings 115C-F include alternating arrays of LEDs and spray nozzles 113 that are physically directed toward the given publicly accessible interface surrounded by the associated application housing 115.

Consistent with that which has been described above, the particular exemplary embodiment shown in the FIG. 5 illustration includes a trigger 107. It is envisioned that the trigger 107 shown in the FIG. 5 illustration may be in the form of a motion detecting sensor, such as an infrared sensor, as would be understood by one of ordinary skill in the art of sensors. Because the application housings 115C-F are stationary by design (as opposed to the hinged application housing 115A shown in previous figures, for example), trigger/sensor 107 may not key off of movement of the application housing 115. And so, it is envisioned that embodiments of the solution may leverage a sensor 107 that detects a user's physical presence in front of dispensing station 101C. When the sensor 107 detects a user presence, it may generate a signal to the controller 105 that, in turn, executes an algorithm according to stored instructions to de-energize the LEDs and/or the spray nozzles 113 if they are active.

Turning now to FIGS. 6A-6C, a more detailed description of the envisioned use for a publicly accessible user interface sanitized by an embodiment of the solution using openly configured application housings, such as application housing 115C, will be described. More specifically, FIGS. 6A-6C demonstrate a method of use for the fuel pump nozzle 405 in the FIG. 5 illustration of the exemplary solution 100.

As can be seen in the FIG. 6A illustration, for example, the “clamshell” application housing 115C includes two raised structures positioned along the length of the fuel pump nozzle 405 when the fuel pump nozzle 405 is stored in its cradle. Referring briefly back to the FIG. 5 illustrations, other application housings 115 may include only a single raised structure and, as such, the number and size and shape and position of the raised structures that form a given application housing 115 are not limiting factors for the scope of the solution.

Returning to the exemplary application housing 115C, each of the two raised structures house arrays of LEDs 111 and spray nozzles 113. In the figure, the LED arrays 111 and spray nozzle arrays 113 are positioned relative to each other such that one single array is created and comprised of alternating LEDs 111 and spray nozzles 113. It is envisioned, however, that other embodiments of the solution may not include a mixture of LEDs 111 and spray nozzles 113 but, rather, one or the other. It is further envisioned that other embodiments of the solution may include a mixture of LEDs 111 and spray nozzles 113 that are arranged differently from the exemplary arrangement depicted in the FIGS. 5-6 illustrations. Moreover, the spray nozzles 113 may be electrostatic 113B or non-electrostatic 113A, as previously described, without departing from the scope of the solution.

In FIG. 6A, both the LED arrays 111 and the spray nozzle arrays 113 are depicted in an active state. That is, LEDs 111 are emitting UVC light and spray nozzles 113 are emitting a fog of liquid chemical disinfectant. As such, it can be understood that the fuel pump nozzle 405 is in the process of getting sanitized. The LEDs 111 and spray nozzles 113 may have been activated as a result of the fuel pump nozzle 405 having been returned to its cradle after use by a user and the sensor 107 determining that the user is no longer in physical proximity to the fuel dispensing station 101C.

In FIG. 6B, it can be understood that the spray nozzles 113 have been deactivated while the LED array 111 continues to emit UVC light. Depending on the embodiment, an algorithm executed via stored instructions in controller 105 may determine that the LED array 111 remains energized when the spray nozzle array 113 is not, and vice versa. Timing and duration and sequence for energizing the arrays 111, 113 will occur to those with skill in the art of pathogen irradiation and remediation.

Finally, in the FIG. 6C illustration, it can be understood that a user is in physical proximity to the fuel dispensing station 101C and, as such, the arrays 111, 113 are de-energized and deactivated. Depending on the embodiment, the arrays 111, 113 may be deactivated in order to provide a user with safe and/or convenient access to the given publicly accessible user interface, such as fuel pump nozzle 405. The fuel pump nozzle 405 is shown as having been removed from its cradle, presumably to be used for replenishing fuel in a tank or container. When the fuel pump nozzle 405 is returned to the cradle, and the sensor 107 determines that the user is no longer physically present, the arrays 111, 113 may be energized and activated in order to sanitize the fuel pump nozzle, as depicted in FIG. 6A and previously described.

Systems and methods for automatically disinfecting publicly accessible user interfaces according to the solution have been described using detailed descriptions of embodiments thereof that are provided by way of example and are not intended to limit the scope of the disclosure. The described embodiments comprise different features, not all of which are required in all embodiments of the solution. Some embodiments of the solution utilize only some of the features or possible combinations of the features. Variations of embodiments of the solution that are described and embodiments of the solution comprising different combinations of features noted in the described embodiments will occur to persons of the art.

It will be appreciated by persons skilled in the art that a system or method for automatically disinfecting publicly accessible user interfaces according to the solution is not limited by what has been particularly shown and described herein above. Rather, the scope of the disclosed solution is defined by the claims that follow.

1-11. (canceled)

12. A system for automatically disinfecting a fuel pump nozzle or a charging cable head for an electric vehicle charging station, the system comprising:
an application housing associated with a fuel pump nozzle or a charging cable head configured to be removably docked in the application housing, wherein:
the application housing comprises one or more stationary, raised structures configured such that the fuel pump nozzle or the charging cable head is accessible to a user when docked in the application housing; and
use of the fuel pump nozzle or the charging cable head by a user requires the user to undock the fuel pump nozzle or the charging cable head from the application housing;
an electrical power source;
a liquid disinfectant reservoir;
one or more arrays of UVC light emitting diodes residing within the one or more raised structures of the application housing and in electrical connection with the electrical power source;
one or more arrays of electrostatic spray nozzles residing within the one or more raised structures of the application housing, the electrostatic spray nozzles being in fluid connection with the disinfectant reservoir and in electrical connection with the electrical power source and operable to generate an electrostatically charged fog of the liquid disinfectant; and
an actuation sensor configured to recognize physical proximity of a user;
wherein when the actuation sensor indicates that a user is in physical proximity to the system the one or more arrays of UVC light emitting diodes and the one or more arrays of spray nozzles are deactivated and when the actuation sensor indicates that a user is no longer in physical proximity to the system, and the fuel pump nozzle or the charging cable head for an is docked in the application housing, the one or more arrays of UVC light emitting diodes are activated in order to subject the fuel pump nozzle or the charging cable head to ultraviolet germicidal irradiation and/or the one or more arrays of the electrostatic spray nozzles are activated in order to apply an electrostatically charged fog of the liquid disinfectant to the fuel pump nozzle or the charging cable head.

13. The system for automatically disinfecting a fuel pump nozzle or a charging cable head for an electric vehicle charging station of claim 12, further comprising:
a controller configured to receive an electrical signal from the actuation sensor and, in response to the signal, cause the one or more arrays to be either activated or deactivated.

14. The system for automatically disinfecting a fuel pump nozzle or a charging cable head for an electric vehicle charging station of claim 13, wherein the controller is a programmable logic controller.

15. The system for automatically disinfecting a fuel pump nozzle or a charging cable head for an electric vehicle charging station of claim 12, further comprising:
a timer component configured to set a duration for which the one or more arrays are activated.

16. The system for automatically disinfecting a fuel pump nozzle or a charging cable head for an electric vehicle charging station of claim 12, further comprising:
a solar charging panel electrically coupled to the power source.

17. The system for automatically disinfecting a fuel pump nozzle or a charging cable head for an electric vehicle charging station of claim 12, wherein the actuation sensor is in the form of an infrared sensor.

18. The system for automatically disinfecting a fuel pump nozzle or a charging cable head for an electric vehicle charging station of claim 12, wherein the one or more arrays of UVC light emitting diodes when activated emit electromagnetic radiation with a wavelength from 10 nm to 400 nm and light with a frequency from 30 petahertz to 750 terahertz.

19. (canceled)

20. (canceled)
